(19) United States
(12) Patent Application Publication
(10) Pub. No.: US 2018/0071025 A1
(43) Pub. Date: Mar. 15, 2018

(54) ORTHOPAEDIC IMPLANT TEMPLATE AND METHOD OF MAKING

(71) Applicant: Biomet Manufacturing, LLC, Warsaw, IN (US)

(72) Inventors: Robert Sixto, Miami, FL (US); Andrea Suarez, Miami Springs, FL (US); Juergen A. Kortenbach, Miami Springs, FL (US)

(21) Appl. No.: 15/818,070

(22) Filed: Nov. 20, 2017

Related U.S. Application Data
(63) Continuation of application No. 14/169,744, filed on Jan. 31, 2014.

(51) Int. Cl. A61F 2/46; C23F 1/02; A61B 17/80; A61F 2/30; A61F 2/42

(52) U.S. Cl. CPC A61B 34/00; A61B 90/06; A61F 2/4657; C23F 1/02; A61F 2/4261; A61B 17/8061; A61F 2/3094; A61F 2002/4658; A61B 2090/061

(57) ABSTRACT

A template for orthopaedic bone implants formed from bio-compatible steel using photo chemical machining and etching. The template is processed on both faces to form right and left guides for implant fasteners and length indicators. Indicia are etched on opposite faces so that it does not overlie one another and presents sufficient contrast to be observed in an x-ray. The manufacturing cost is sufficiently low that the template may be provided in a single use form.

### BACKGROUND OF THE INVENTION

### 1. Field of the Invention

The present invention relates to orthopaedic devices, and, more specifically, to templates and methods of making same for orthopaedic implants.

### 2. Description of the Related Art

The field of orthopaedics has been towards sterile options regarding the equipment used during surgical procedures. This trend has as its objective, lower risk of infection, insured product integrity and potential improvement efficiency of care.

One aspect experiencing increased attention is in the area of a template or trial that is used for sizing orthopaedic implant devices used to repair a trauma or other problem with bone material. The templates are used to select the proper size implant during a surgical procedure to avoid contamination of a very expensive implant device, if it proves to be not the right size for a particular application. Currently, such templates are manufactured from machined, stainless steel or a 1000 grade aluminum templates that are anodized. Both template types are relatively expensive but less costly than the implant. With the trend toward a total sterile environment for orthopaedics, the existing templates require re-sterilization and indefinite use, thereby increasing the total cost of the medical procedure.

What is needed in the art therefore is an orthopaedic implant template that is manufactured with sufficiently reduced cost to economically justify a one use option for the template.

### SUMMARY OF THE INVENTION

The present invention provides a low cost template with sufficient economy of manufacturing to enable a single use.

In one form, the invention is an orthopaedic template formed from a metal sheet by photo chemical machining to cut through the metal sheet for the template outline, hole positions and length marks. The thin metal sheet is also photo etched to provide product information.

In another form, the invention is a method of making an orthopaedic implant template from a metal sheet by applying a photo sensitive sheet to at least one face of the metal sheet. A photo negative pattern is supplied to the photo sensitive sheet with the pattern being configured to the outline of the template and to any openings. The sheet is exposed to a light source to harden the exposed pattern and the non-hardened portions are chemically removed to form the final pattern. The resultant sheet metal is etched to form the outline of the templates and the openings.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a side view of one face of a template embodying the present invention;

FIG. 2 is a side view of an opposite face of the template of FIG. 1;

FIG. 3 shows an array of different size templates; and

FIG. 4 is a simulation of an x-ray showing a template embodying the present invention.

Corresponding reference characters indicate corresponding parts throughout the several views. The exemplifications set out herein illustrates one embodiment of the invention and such exemplifications is not to be construed as limiting the scope of the invention in any manner.

### DETAILED DESCRIPTION OF THE INVENTION

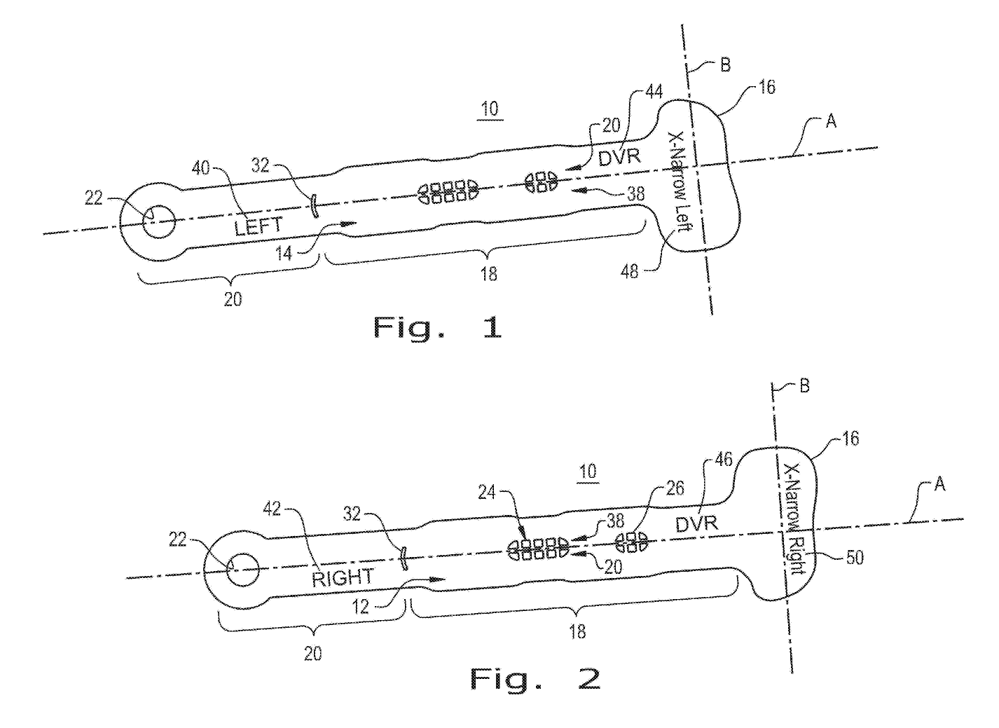

Referring specifically to FIGS. 1 and 2, there is shown a template 10 formed according to the present invention. The template 10 has a right face 12 and left face 14 shown in FIGS. 2 and 1 respectively. The template 10 has a proximal section 16 having a configuration that represents the outline of an implant to be used in orthopaedic surgery. The shape of proximal section 16 is simply illustrative of one of many shapes that may be employed for this purpose. A distal section 18 is integral with the proximal section 16 and represents the portion of an implant that is fastened to bone material. A base section 20 is integral with and extends from distal section 18 and terminates with a opening 22 to be used in connection with an array of templates 10, as described below.

The template 10 has a longitudinal axis A and an additional reference axis B extending tangentially with respect to axis A within the proximal section 16. A plurality of holes 24 and 26 are provided in the proximal section 18. As observed in FIGS. 1 and 2 the holes 24 and 26 have an upper portion 28 and lower portion 30 symmetrical with respect to longitudinal axis A. As illustrated between FIGS. 1 and 2, the relationship of the holes to the longitudinal axis reverses with respect to the right and left faces 12 and 14. A curved slot 32 is formed at the end of the distal section 18 and forms an indicator of the length of the implant the template 10 would represent. The plurality of holes 24 and 26 allow flexibility in the positioning of fasteners that are used to secure in place the implant the template 10 is intended to represent. By having multiple openings, flexibility in the placement of implant fasteners is accommodated, taking into account the location of any bone fracture along the length of the bone.

The template 10 is also provided with informational indicia on both faces 12 and 14 of the template 10. These indicia include whether it is for a right or left bone as indicated at 40 or right 42. It should be noted that the FIGS. 1 and 2 show the face of the template 10 and that the left indicator is below axis A on face 14 and below the axis A on face 12 so that in practice the indicators 40 and 42 do not overlie one another. The same is true of the DVR indicator 44 and 46. The indica 44 and 46 also do not overlie one another. The proximal section 16 also has indicators identified as X-narrow left by reference number 48 and X-narrow right indicated by reference character 50. In this case, the reference indicators 48 and 50 are staggered with respect to one another and on opposite sides of reference line B so that they do not overlie one another. The feature of not having the indicia overlie one another will become apparent in the description of the formation of the template 10.

In accordance with another aspect of the present invention, the template 10 is formed by photo chemical machining and photochemical etching. This process enables significant reductions in manufacturing costs and reduces the unit cost of template 10 to the point where it is economically feasible to provide the template 10 in a one use package. The process begins with providing a sheet of bio-compatible metallic material, typically stainless steel used in surgery. A photo sensitive sheet with an adhesive back is rolled preferably on both sides of the sheet and then a machine loads a photo negative of the material of the ultimate configuration in a glass press and this is done on both sides. Subsequently, both sides are exposed to a light source such as ultraviolet to harden the exposed portion of the template configuration, namely what is contained outside of the negative. The material that has not been hardened is then stripped by a bath which immerses the sides in a chemical that strips the exposed areas away, leaving the configuration of the part as shown in FIGS. 1-3. The sheet material is then etched in a system which uses high pressure acid traversing the sheet to remove the portions of the material not covered by the hardened photo sensitive material. Typically, on each side, the etching extends to about 60% of the thickness of the material. When it is done to opposite sides, it cuts through the material for the outline and for the holes 24 and 26 for screw placement and slot 32 for length determination. Subsequent to the photo etching process, the photo sensitive material is stripped from the sheet and then the entire finished part is subjected to a brief acid etching to remove sharp edges that may be a cutting hazard.

On the portion of the template on which the indicia regarding information is placed, the etching is only on one side, since the etching goes through more than half of the thickness of the template 10. Accordingly, the photo negative used to manufacture the template 10 is set up so that the indicia are positioned on opposite sides of the longitudinal axis A and the transverse axis B to prevent any breakthrough, while providing sufficient depth to the indicia. This enables sufficient optical contrast to easily read the various indicia placed on the template 10.

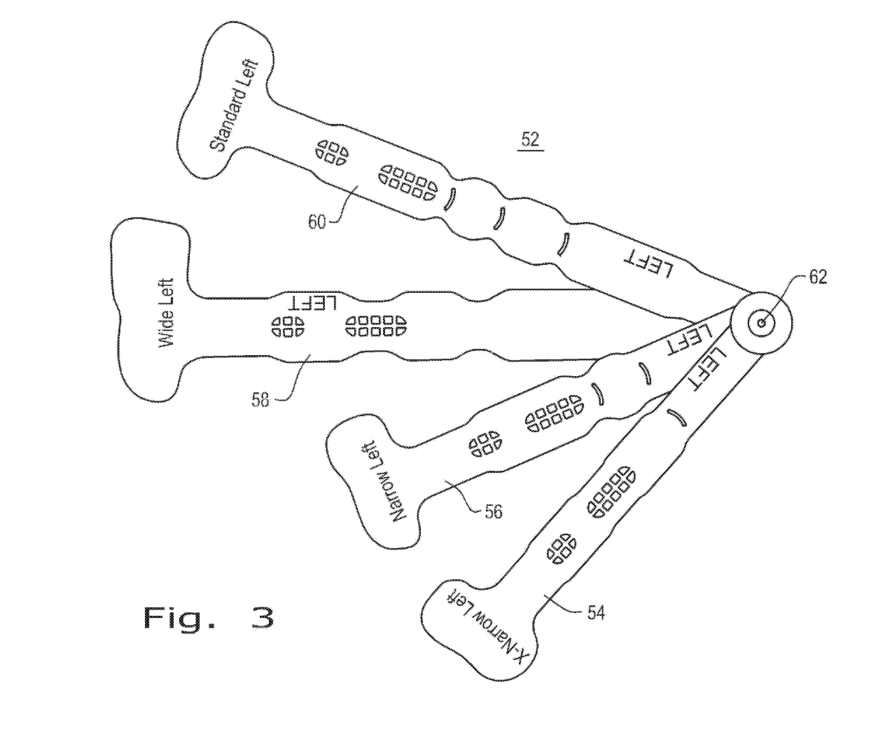

The templates 10 may be placed in an array 52 shown in FIG. 3, which shows a plurality of templates 54, 56, 58 and 60 all secured through opening 10 by a fastener 62. As is apparent from FIG. 3, the array 52 as viewed in FIG. 3 is of the left side. The difference in the length of the elements has to do with the size as indicated on the face. The faces opposite to that shown in FIG. 3 would be for the right side so that the array 52 of FIG. 3 shows eight possibilities of templates. This allows an economy of scale that is heretofore not been achieved.

The thickness of the templates 10 can be up to 1.5 mm and preferably is 0.3 mm. They are formed from stainless steel suitable for surgical applications. The thickness is thick enough for the lettering on the indicia to show but thin enough to flex so that they may be bent to conform to the bone structure. Preferably, they are made from spring tempered material so that they may be reused if necessary.

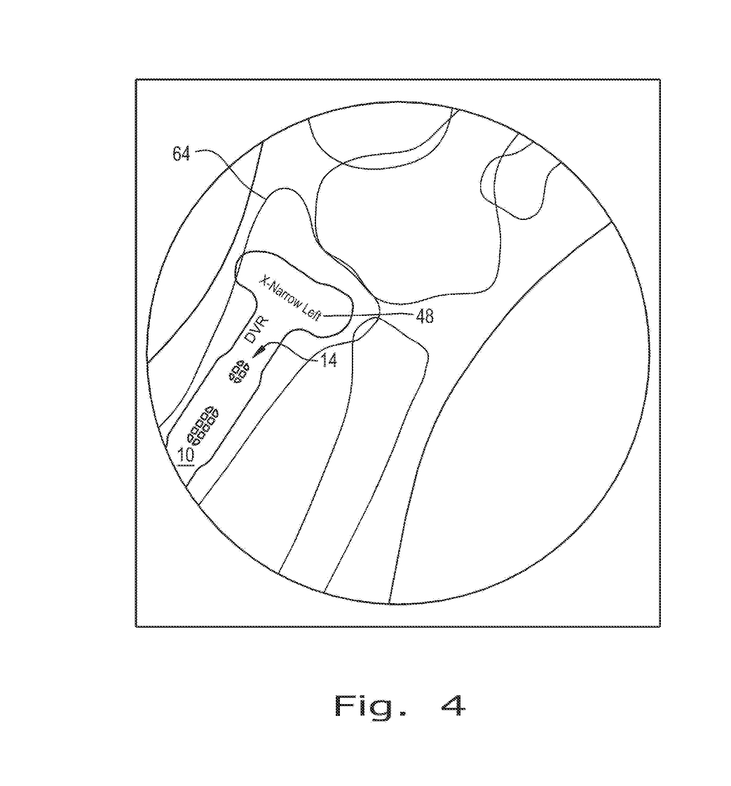

The templates will in FIG. 4 which is an x-ray of a healthy bone 64 with the template 10 placed in the x-ray field. The template 10 shows the left side 14 and the difference in thickness of the template 10 at the indicia 48 is sufficient to allow the indicia to show up in the x-ray. This is an added benefit for the surgeon who has a ready reference of the particular template that was used.

The use of photo chemical machining and etching to manufacture the templates 10 provides a cost of manufacturing that is sufficiently reduced to make the templates 10 available in a one use application. For this application, a template 10, or an array of templates would be prepackaged and sterilized by subjecting them to gamma rays, for example.

In contrast to previous methods of machining and forming expensive materials, the photo chemical machining and etching enables a significant flexibility without the excess manufacturing cost associated with prior methods. In addition, the templates 10, because they are etched on both sides, allow a right and left presentation in a single element. Because the templates 10 are made of spring steel, they may flex to accommodate an orthogonal projection of the implant they represent. In this case, the shape of the template is not the actual configuration of the implant but a projection of the shape onto a plane that is not parallel to the plane of the implant. This feature enables a surgeon to see in an X-ray or other image if the actual template is the appropriate size when muscle tissue and other body components proven presentation of the template in the X-ray in a plane parallel to the plane of the implant in the human anatomy. In addition, the template 10 may have indications of the trajectory of the screws fastening the implant to a bone. As one example of this purpose, a pair of tabs 66 and 68 (show by dashed lines in FIG. 1) represent the path of the screws as viewed from the perspective of the image. This enables a surgeon to determine whether the actual implant selected will fit and be secured to the proper bone element.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1-20. (canceled)

21. A method of making an orthopaedic implant template that is flexible to conform to human anatomy, the method comprising:
providing, receiving or forming a thin metal sheet having a first face and a second face opposite the first face and a thickness extending therebetween;
photochemical machining the thin metal sheet to cut an implant template outline; and
photochemical etching product information on at least one face of the thin metal sheet.

22. The method of claim 21, wherein the implant template outline is not an actual configuration of an orthopaedic implant, and wherein the photochemical machining the implant template outline includes photochemical etching the first face and the second face to cut through the thin metal sheet.

23. The method of claim 21, wherein the photochemical etching extends through up to 60% of the thin metal sheet.

24. The method of claim 21, wherein the photochemical etching product information includes photochemical etching a first product information on the first face and a second product information on the second face, and wherein the first product information is etched in a location offset from the second product information such that the first product information and the second product information do not overlie one another.

25. The method of claim 21, further comprising:
providing the orthopaedic implant template in a plurality of sizes; and
pivotally connecting the plurality of sizes of the orthopaedic implant template to each other to form an array of sizes.

26. The method of claim 21, wherein the thickness of the thin sheet is up to 1.5 mm to provide flexibility to conform to the human anatomy.

27. The method of claim 21, further comprising photochemical machining the thin metal sheet to cut a plurality of holes and one or more length marks.

28. The method of claim 21, further comprising photochemical machining the thin metal sheet to cut an opening in a base section.

29. The method of claim 21, wherein the thickness of the thin metal sheet is 3.0 mm to provide flexibility to conform to the human anatomy.

30. The method of claim 21, wherein photochemical machining and photochemical etching includes:
applying a photo sensitive sheet including a photo sensitive material to at least one of the first face or second face of the thin metal sheet;
applying a photo negative pattern to the photo sensitive sheet, the photo negative pattern corresponding to the implant template outline;
exposing the photo sensitive sheet to a light source to expose and create a hardened portion of the photo sensitive material not covered by the photo negative pattern, and leaving the photo sensitive material that is covered by the photo negative pattern as an unhardened portion;
chemically stripping the unhardened portion of the photo sensitive material from the thin metal sheet;
cutting the implant template outline by chemically removing a portion of the thin metal sheet where the unhardened portion was removed, wherein cutting includes cutting through a thickness of the thin metal sheet from the first face to the second face;
chemically etching through a portion of the thickness of the thin metal sheet to etch product information on at least one side of the thin metal sheet; and
chemically removing the hardened portion of the photo sensitive material from the thin metal sheet.

31. A method of making an orthopaedic implant template from a thin metal sheet that is flexible to conform to human anatomy, the method comprising:
providing, receiving or forming a thin metal sheet having a first face and a second face opposite the first face and a thickness extending therebetween;
applying a photo sensitive sheet including a photo sensitive material to at least one of the first face or the second face of the thin metal sheet;
applying a photo negative pattern to the photo sensitive sheet, the photo negative pattern corresponding to an implant template outline;
exposing the photo sensitive sheet to a light source to expose and create a hardened portion of the photo sensitive material not covered by the photo negative pattern, and leaving the photo sensitive material that is covered by the photo negative pattern as an unhardened portion;
chemically stripping the unhardened portion of the photo sensitive material from the thin metal sheet;
cutting the implant template outline by chemically removing a portion of the thin metal sheet where the unhardened portion was removed, wherein cutting includes cutting through a thickness of the thin metal sheet from the first face to the second face;
chemically etching through a portion of the thickness of the thin sheet to etch product information on at least one side of the thin metal sheet; and
chemically removing the hardened portion of the photo sensitive material from the thin metal sheet.

32. The method of claim 31, wherein the cutting through the thickness of the thin metal sheet includes cutting through the thickness of up to 1.5 mm.

33. The method of claim 31, wherein cutting the implant template outline includes cutting the implant template outline in a proximal section of the orthopaedic implant template, wherein the implant template outline is not an actual configuration of an orthopaedic implant.

34. The method of claim 31, wherein the photo negative pattern includes a plurality of holes and one or more length marks.

35. The method of claim 31, wherein the photo negative pattern includes an opening in a base section of the orthopaedic implant template.

36. The method of claim 31, wherein chemically removing a portion of the thin metal sheet where the unhardened portion was removed includes chemically etching the first face and the second face to cut through the thin metal sheet.

37. The method of claim 31, wherein the chemically removing extends through up to 60% of the thin metal sheet.

38. The method of claim 31, wherein the chemically etching the product information includes chemically etching a first product information on the first face and a second product information on the second face, and wherein the first product information is etched in a location offset from the second product information such that the first product information and the second product information do not overlie one another.

39. The method of claim 31, further comprising providing the orthopaedic implant template in a plurality of sizes and pivotally connecting the plurality of sizes of the orthopaedic implant template to each other to form an array of sizes.

40. The method of claim 31, wherein cutting the implant template outline includes cutting through a portion of the thickness on both the first face and the second face to form the implant template outline.
